(19) United States
(12) Patent Application Publication
(10) Pub. No.: US 2022/0234041 A1
(43) Pub. Date: Jul. 28, 2022

(54) INTEGRATED SENSOR ARRAY AND CIRCUITRY

(71) Applicant: Analog Devices International Unlimited Company, Limerick (IE)

(72) Inventors: Alain Valentin Guery, Boston, MA (US); Hari Chauhan, Windham, NH (US); Mohamed Azize, Medford, MA (US); Joyce H. Wu, Somerville, MA (US); Olive H. Murphy, Macroom (IE); Craig Alan Breen, Arlington, MA (US); J Brian Harrington, Revere, MA (US)

(21) Appl. No.: 17/615,472

(22) PCT Filed: Jul. 7, 2020
(86) PCT No.: PCT/US2020/041040

§ 371 Date: Nov. 30, 2021

Related U.S. Application Data
(60) Provisional application No. 62/871,338, filed on Jul. 8, 2019.

(51) Int. Cl. B01L 3/00; G01N 27/414

(52) U.S. Cl. CPC B01L 3/502715; G01N 27/4145; G01N 27/4146; G01N 27/4148; B01L 2300/0896; B01L 2300/0636; B01L 2300/0663; B01L 2300/0819; B01L 2300/126; B01L 2200/12

(57) ABSTRACT

Sensors having dimensions on the order of nanometers can be arranged in an array. The sensors can detect substances found in an environment. The array of sensors can be disposed on a substrate along with circuitry to control the operation of the array of sensors.

### CLAIM OF PRIORITY

This patent application claims the benefit of priority to U.S. Provisional Application Ser. No. 62/871,338, filed Jul. 8, 2019, which is incorporated by reference herein in its entirety.

### FIELD OF THE DISCLOSURE

This document pertains generally, but not by way of limitation, apparatuses and methods related to sensory arrays.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 is a block diagram depicting an example device that includes an integrated sensor array and circuitry.

FIG. 2 is a block diagram depicting a system that includes a device including another example of an integrated sensor array and circuitry in addition to a sensor reader device and sensor software.

FIG. 3 is a conceptual diagram depicting a sensor that can be included in an integrated sensor array and circuitry.

FIG. 4A is a conceptual diagram depicting detection of a substance by contacting the substance with at least one sensor of a sensor array included in a device.

FIG. 4B is a conceptual diagram depicting detection of a substance by at least one sensor of a sensor array included in a device without contacting the substance with the at least one sensor.

FIG. 5 is a flow diagram depicting operations of a process to produce and use an integrated sensor array and circuitry.

### DETAILED DESCRIPTION

Nanosensors are devices that are capable of detecting amounts of substances in an environment. Nanosensors can detect gas particles, liquid particles, and/or solid particles where the sizes of the particles can be on the order of 10⁻⁹ m or less. Typically, nanosensors have dimensions on the order of 750 nm or less and can detect various substances, such as biological substances, found within an organism. For example, nanosensors can detect the presence of biological substances found in the blood and/or urine of individuals, such as glucose, lactose, metals, proteins, volatile organic compounds (VOCs), biomarkers, microorganisms, genetic molecules (e.g., DNA, RNA), and the like. Additionally, nanosensors can detect the presence of particles found in the air or other gaseous environments, such as pollutants or other particulates. Further, nanosensors can detect the presence of substances in solids, such as food products, to identify the composition of the solids or to identify contaminants in the solids.

Nanosensors can include materials that have specified chemical properties, mechanical properties, electrical properties, acoustic properties, or optical properties that are responsive to changes in an environment that can be caused by the presence or absence of various particles within the environment. For example, nanosensors can be sensitive to chemical reactions, heat, mechanical stress, changes in concentration, volumetric changes, gravitational forces, magnetic forces, and/or electrical forces. In response to stimulation generated by the presence of one or more substances in an environment, nanosensors can produce one or more signals, such as electrical signals, that indicate the presence or concentration of the substance in the environment. To illustrate, nanosensors can be contact sensors that are responsive to contact by one or more substances. In additional implementations, nanosensors can be non-contact sensors that measure optical properties of substances or an environment to detect the presence of a substance. In one or more examples, nanosensors can have a relatively high level of sensitivity and can detect relatively small amounts of substances in an environment, such as on the order of nanograms per milliliter (mL) down to picograms per mL.

Advances have been made in the reduction of the size of nanosensors, the reliability of nanosensors, and the number of substances detected by nanosensors. However, integration of nanosensors with circuitry to control the nanosensors and with circuitry enabling nanosensors to interface with electronic devices is still needed.

This disclosure describes an array of sensors that is integrated with circuitry that can be used in relation to the control and operation of the sensors included in the array of sensors. The sensors included in the array can detect particles having sizes on the order of 10⁻⁹ m. The sensors included in the array of sensors can also have dimensions on the order of no greater than about 10 micrometers, no greater than about 8 micrometers, no greater than about 5 micrometers, no greater than about 3 micrometers, no greater than about 1 micrometer, or no greater than about 750 nm. Additionally, this disclosure describes circuitry that can be configured to communicate signals produced by the sensors to one or more electronic devices that can analyze the signals. The array of sensors can be disposed on a substrate and circuitry that can be configured with respect to the control and operation of the sensors can also be disposed on the same shared substrate. In various implementations, semiconductor manufacturing processes can be used to form the circuitry on the substrate. Further, connectors can be disposed on the substrate that electrically couple the array of sensors to the circuitry. For example, the connectors can carry one or more signals to the sensors disposed on the substrate and the connectors can enable one or more signals from the sensors to be captured, stored, and analyzed. In various implementations, circuitry to couple the sensor array to a data reader device can also be disposed on the substrate. In one or more implementations, the data reader device can obtain one or more signals from the sensor array and can provide the signals to one or more analytics platforms that can be used to analyze the signals and to provide the results of the analysis to a user of the one or more analytics platforms.

In one or more examples, the sensors used in implementations herein can include a variety of sensing elements. For example, the sensors can include semiconductor devices, such as field effect transistors (FETs). In one or more additional examples, the sensors can include carbon nanotubes (CNTs). Further, the sensors can include wires, such as Au (gold) or Pt (platinum) wires that can have diameters that are less than about 250 nm. In illustrative implementations, the substrates on which the sensors and the circuitry are disposed can be relatively rigid substrates, such as silicon-containing substrates or glass-containing substrates. In other illustrative implementations, the substrates on which the sensors and circuitry are disposed can be relatively flexible. To illustrate, the sensors and the circuitry can be disposed on a polymeric substrate, such as a polyamide-containing substrate or a polyethylene terephthalate-containing substrate.

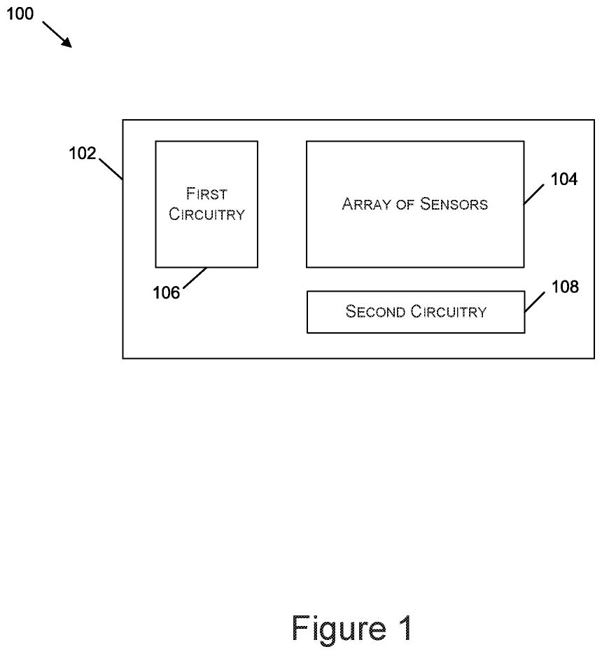

FIG. 1 is a block diagram depicting an example device 100 that includes an integrated sensor array and circuitry in accordance with this disclosure. For example, the device 100 includes a substrate 102. The substrate 102 can be relatively rigid, in some implementations, while in other examples, the substrate 102 can be relatively flexible. The substrate 102 can be formed from a silicon-containing or semiconductor material, in various implementations. Additionally, the substrate 102 can be formed from a glass-containing material. In further implementations, the substrate 102 can be formed from a polymeric material. To illustrate, the substrate 102 can be formed from a polyamide-containing material. The substrate 102 can also be formed from a polyethylene terephthalate-containing material. In still other examples, the substrate 102 can be formed from a paper-containing material, such as cellulose. In one or more illustrative examples, the substrate 102 can have a number of layers with at least one layer comprised of a material that is different from a material of another layer. For example, the substrate 102 can include a silicon-containing substrate with one or more oxide layers disposed on the silicon-containing substrate. The one or more oxide layers can comprise silicon dioxide. Further, in one or more examples, the substrate 102 can comprise a plurality of layers of a same material.

An array of sensors 104 can be disposed on the substrate 102. The array of sensors 104 can include a number of sensors, e.g., on the order of tens of sensors to on the order of thousands of sensors. The individual sensors included in the array of sensors 104 can be arranged in a pattern such as in a grid having a number of rows and a number of columns. The individual sensors included in the array of sensors 104 can include sensors that can have two terminal elements and that can be formed from one or more carbon nanotubes (CNTs).

In one or more additional implementations, the individual sensors included in the array of sensors 104 can include wires or other electrically conductive traces, such as having multiple electrodes and having diameters (or other cross-sectional dimension) no greater than 250 nm, no greater than 200 nm, no greater than 150 nm, no greater than 100 nm, or no greater than 50 nm. In various implementations, wires forming or included in the individual sensors included in the array of sensors 104 can have a diameter from 10 nm to 250 nm, from 20 nm to 150 nm, or from 25 nm to 100 nm, inclusive. In one or more illustrative examples, the wires comprising or included in the individual sensors included in the array of sensors 104 can include at least one of gold-containing wires or wires including a gold alloy. The wires of the individual sensors included in the array of sensors 104 can also include at least one of platinum-containing wires or wires including a platinum alloy. In one or more additional examples, the wires of the individual sensors included in the array of sensors 104 can include carbon-containing wires. In various examples, wires of the individual sensors included in the array of sensors can include CNTs. The CNTs can also be formed into structures other than wires, such as sheets of CNTs.

In one or more further implementations, the individual sensors included in the array of sensors 104 can include a field effect transistor (FET) structure. In various examples, the FET structure of the individual sensors included in the array of sensors can include at least one of a finFET structure, an ion-sensitive FET structure, or a bioFET structure. Further, the individual sensors included in the array of sensors 104 can include an organic FET. The organic FETs can have channels that can include one or more organic semiconductor materials. In one or more illustrative examples, the individual sensors included in the array of sensors 104 can include n-type zinc oxide-containing field effect transistors. Additionally, the individual sensors included in the array of sensors 104 can include at least one of p-type silicon-containing field effect transistors, n-type silicon-containing field effect transistors, p-type germanium-containing transistors, or n-type germanium-containing transistors.

The device 100 can also include first circuitry 106 disposed on the substrate 102 and second circuitry 108 disposed on the substrate 102. The first circuitry 106 and the second circuitry 108 can include semiconductor devices disposed on the substrate 102. In one or more illustrative examples, at least one of the first circuitry 106 or the second circuitry 108 can include field effect transistors. Additionally, the first circuitry 106 and the second circuitry 108 can include connectors disposed on the substrate 102. The connectors can include metal traces that can be used to carry signals between sensors included in the array of sensors 104 and components of the first circuitry 106 and the second circuitry 108. The first circuitry 106 and the second circuitry 108 can also include a number of switches that can be operated to activate and deactivate sensors included in the array of sensors 104. Further, the first circuitry 106 and the second circuitry 108 can include at least one of digital circuitry or analog circuitry such as at least one of registers, gates, D-flip-flops, inverters, current mirror circuitry, resistors, capacitors, or amplifiers. In various implementations, features of at least one of the first circuitry 106 or the second circuitry 108 can comprise carbon nanotubes.

The first circuitry 106 and the second circuitry 108 can control the operation of the individual sensors included in the array of sensors 104. For example, the first circuitry 106 and the second circuitry 108 can control the activation and/or the deactivation of individual sensors included in the array of sensors 104. The sensors included in the array of sensors 104 can be in an activated state when the sensors are capable of detecting the presence of a substance and communicating an indication of the presence of the substance to at least one of the first circuitry 106 or the second circuitry 108. In addition, a sensor of the array of sensors 104 can be in a deactivated state when the sensor is unable to detect the presence of a substance and unable to communicate the indication of the presence of that substance to at least one of the first circuitry 106 or the second circuitry 108.

In one or more implementations, a sensor included in the array of sensors 104 can be activated when one or more connectors coupled to at least one of the first circuitry 106 or the second circuitry 108 are enabled to carry a signal between the sensor to at least one of the first circuitry 106 or the second circuitry 108. In various examples, switches included in at least one of the first circuitry 106 or the second circuitry 108 can be operated to activate sensors of the array of sensors 104. For example, electrical signals can respectively be applied to sensors of the sensor array 104 to cause the corresponding sensors to be in activated state. Further, the sensors of the sensor array 104 can be in a deactivated state when the sensors are not in electrical communication with at least one of the first circuitry 106 or the second circuitry 108. In one or more illustrative examples, switches included in at least one of the first circuitry 106 or the second circuitry 108 can be operated to deactivate sensors of the sensor array 104. The deactivation of sensors included in the sensor array 104 can take place through the absence of corresponding electrical signals being provided to the respective sensors via at least one of the first circuitry 106 or the second circuitry 108.

The first circuitry 106, the second circuitry 108, or both the first circuitry 106 and the second circuitry 108 can include components to store signals obtained from sensors included in the sensor array 104. To illustrate, at least one of the first circuitry 106 or the second circuitry 108 can include memory circuitry to store data indicating the presence and/or concentration of substances detected by sensors included in the sensor array 104. Additionally, at least one of the first circuitry 106 or the second circuitry 108 can include one or more components to communicate information to one or more additional devices via one or more networks. In various examples, at least one of the first circuitry 106 or the second circuitry 108 can include network interfaces or other communications ports, such as that enable wireless communication, wired communication, or communication by physically coupling the device 100 to another device.

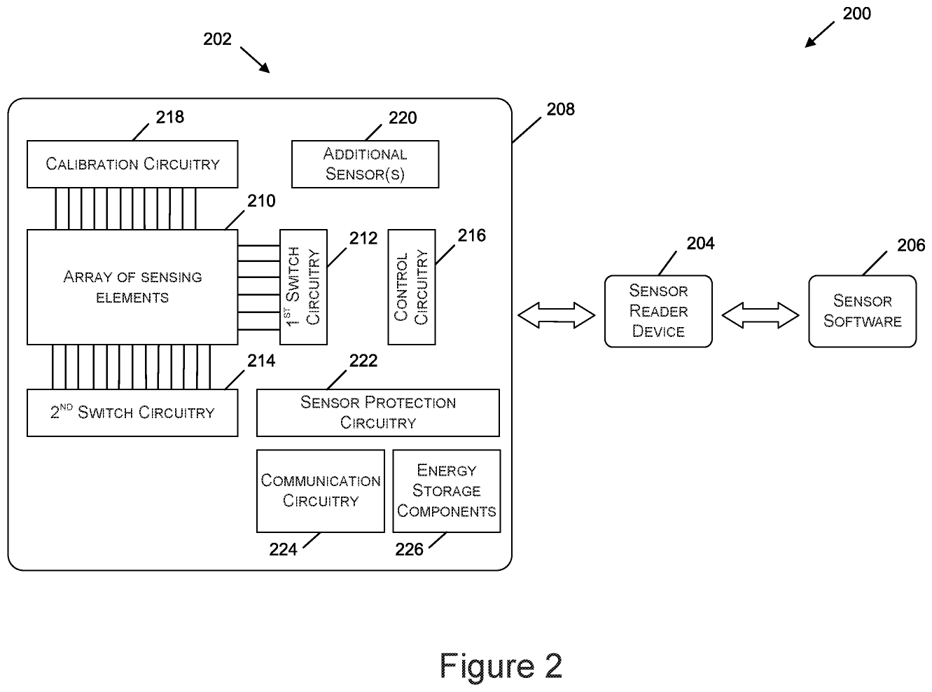

FIG. 2 is a block diagram depicting a system 200 that includes a device 202 comprising another example of an integrated sensor array and circuitry in addition to a sensor reader device 204 and sensor software 206 in accordance with this disclosure. The device 202 can include a substrate 208 and an array of sensing elements 210 disposed on the substrate 208. In implementations, the substrate 208 can be the same as or similar to the substrate 102 described with respect to FIG. 1 and the array of sensing elements 210 can be the same as or similar to the array of sensors 104 described with respect to FIG. 1. Thus, the substrate 208 can have a composition and structure that is the same or similar to that described with respect to the substrate 102 described with respect to FIG. 1 and the array of sensing elements 210 can have a same or similar composition and structure as the array of sensors 104 described with respect to FIG. 1.

Individual sensing elements of the array of sensing elements 210 can be functionalized such as to detect the presence of one or more substances. That is, individual sensing elements of the array of sensing elements 210 can have a structure and/or be formed from materials that cause the individual sensing elements to be predisposed toward the detection of one or more specified substances. In various implementations, the individual sensing elements of the array of sensing elements 210 can include a chemical filter that can detect the presence of a substance. The chemical filter can filter molecules having particular chemical and/or physical properties to be sensed by other portions of the nanosensing elements, such as sensing circuitry. In illustrative examples, the array of sensing elements 210 can include sensing elements that can detect molecules in the blood of an individual, such as glucose or lactose. In additional examples, the array of sensing elements 210 can include sensing elements that can detect substances in the air, such as pollutants or other particulate matter. The array of sensing elements 210 can also detect substances found in solids, such as powders.

Individual sensing elements included in the array of sensing elements 210 can also detect the presence of electrical activity that can indicate an amount of a substance within a sample. For example, chemical reactions that take place in the presence of at least one of one or more reactants or one or more catalysts can produce an electrical response that is detectable by individual sensing elements of the array of sensing elements 210. That is, various chemical reactions can produce electrons in an amount that is proportional to the concentration of a substance within a sample. In these scenarios, by measuring the amount of electrical activity that takes place with respect to a sample, an amount of a substance included in the sample can be determined.

In one or more additional implementations, the array of sensing elements 210 can include a number of individual sensing elements that can detect the presence of electromagnetic radiation having one or more ranges of wavelengths. In these scenarios, the array of sensing elements 210 can include or be disposed in relation to an array of emitter elements. In one or more examples, individual emitter elements can produce electromagnetic radiation having at least one range of wavelengths and detecting elements included in the array of sensing elements 210 can detect changes to the emitted electromagnetic radiation. The changes to the emitted electromagnetic radiation in relation to the detected electromagnetic radiation can be caused by one or more substances included in a sample. In one or more illustrative examples, substances can produce characteristic signatures of electromagnetic radiation intensities with respect to a number of wavelengths. In these instances, data obtained from the emitting elements and the detecting elements of the array of sensing elements can be analyzed. The analysis can include analyzing a profile of detected electromagnetic radiation detected by one or more sensing elements of the array of sensing elements 210 with respect to predetermined profiles indicating intensity of electromagnetic radiation detected with respect to a number of wavelengths for respective substances. In various examples, the analysis can determine an amount of similarity between an electromagnetic radiation profile detected by the array of sensing elements 210 and one or more previously determined electromagnetic radiation profiles for one or more substances. The presence of a substance can be identified based on the amount of similarity being at least a threshold amount of similarity for a respective substance.

The array of sensing elements 210 can include from tens to hundreds up to thousands of sensing elements that can individually detect the presence of one or more substances. The individual sensing elements included in the array of sensing elements 210 can be arranged in a desired manner, such as in a grid along a number of rows and a number of columns, such as an N×M matrix of sensing elements. In particular examples, the array of sensing elements 210 can include at least one sensing element that detects a first substance and at least one sensing element that detects a second, different substance. In this way, the array of sensing elements 210 can detect the presence of multiple different substances when placed in a same environment or in different environments. Additionally, the array of sensing elements 210 can include multiple sensing elements that can detect a same substance or a same set of substances. Accordingly, the array of sensing elements 210 can include redundant sensing elements to detect the presence of a substance or to detect the presence of a set of substances. In various implementations, the number of sensing elements included in the array of sensing elements 210 that are configured to detect a substance can be based on a reliability and/or accuracy of the sensing elements to detect the substance. For example, in situations where sensing elements used to detect a substance have at least a threshold reliability and/or a threshold accuracy, the array of sensing elements 210 can include fewer sensing elements to detect the substance in relation to scenarios where sensing elements used to detect the substance have less than the threshold reliability and/or less than the threshold accuracy. In one or more implementations, sensing elements that detect a substance can be grouped together within the array of sensing elements 210, while in other implementations, sensing elements that detect a particular substance can be located at different locations within the array of sensing elements 210.

Further, the number of sensing elements included in the array of sensing elements 210 that are configured to detect a substance can be based on an expected lifetime of the individual sensing elements and an amount of time that the array of sensing elements 210 is expected to be used to detect substances in an environment. To illustrate, as the expected lifetime of sensing elements decreases, an increasing number of the sensing elements can be included in the array of sensing elements 210. Further, as the time that sensing elements are expected to be activated within an environment increases, an increasing number of the sensing elements can be included in the array of sensing elements 210.

First switch circuitry 212 and second switch circuitry 214 can be disposed on the substrate 208. The first switch circuitry 212 and the second switch circuitry 214 can include a number of switches that are coupled with the sensing elements included in the array of sensing elements 210. In particular implementations, individual rows of sensing elements included in the array of sensing elements 210 can be coupled to individual switches included in the first switch circuitry 212. Additionally, individual columns of sensing elements included in the array of sensing elements 210 can be coupled to individual switches included in the second switch circuitry 214. Switches included in the first switch circuitry 212 and the second switch circuitry 214 can be implemented as semiconductor devices. In one or more additional examples, switches included in the first switch circuitry 212 and the second switch circuitry 214 can be implemented as carbon nanotubes. In one or more implementations, at least one of the first switch circuitry 212 or the second switch circuitry 214 can include shift register circuitry coupled to the switches included in the first switch circuitry 212 and/or the second switch circuitry 214. The shift register circuitry can be used to control the operation of rows and/or columns of the array of sensing elements 210.

Control circuitry 216 can also be disposed on the substrate 208. The control circuitry 216 can control the operation of switches and register circuitry included in the first switch circuitry 212 and the second switch circuitry 214. In one or more implementations, the control circuitry 216 can cause switches included in the first switch circuitry 212 and the second switch circuitry 214 to operate by being in an open state or in a closed state. By causing the switches included in the first switch circuitry 212 and the second switch circuitry 214 to operate in an open state or a closed state, the control circuitry 216 can cause electrical signals to be communicated to or communicated from sensing elements included in the array of sensing elements 210.

In one or more illustrative implementations, the sensing elements of the array of sensing elements 210 can be arranged such that each sensing element is located at an intersection of a first connector extending along a column of sensing elements and a second connector that is extending along a row of sensing elements. In these scenarios, to enable electrical signals to be received by and sent to an individual sensing element, the control circuitry 216 can cause a first switch that is coupled to the sensing element via the first connector and a second switch that is coupled to the sensing element via the second connector to close. In some implementations, the closing of the switches in the first switch circuitry 212 and the second switch circuitry 214 that are coupled to an individual sensing element can cause the sensing element to be in an activated state. Further, the opening of the switches in the first switch circuitry 212 and the second switch circuitry 214 coupled to an individual sensing element can cause the sensing element to be in a deactivated state.

Calibration circuitry 218 can also be disposed on the substrate 208. The calibration circuitry 218 can determine one or more operating conditions for individual sensing elements included in the array of sensing elements 210. For example, the signals produced by individual sensing elements included in the array of sensing elements 210 can generate signals with different characteristics in response to the detection of a substance. To illustrate, a first sensing element of the array of sensing elements 210 can generate a signal having a first set of characteristics in response to detecting a substance and a second sensing element of the array of sensing elements 210 can generate a signal having a second set of characteristics that is different from the first set of characteristics in response to detecting the substance. In one or more illustrative examples, a voltage change that takes place with respect to the first sensing element in response to detection of a substance can be different from a voltage change that takes place with respect to the second sensing element in response to detection of the substance. In these situations, the presence of a substance can be indicated by different voltage changes at the different sensing elements. Thus, relying on the same threshold voltages to determine whether an individual sensing element has detected the presence of a substance can lead to false positives or false negatives. Accordingly, the calibration circuitry 218 can determine individual baseline sets of characteristics for the individual sensing elements included in the array of sensing elements 210 to set thresholds for determining when the individual sensing elements detect a substance.

Further, additional sensors 220 can be disposed on the substrate 208. For example, sensors other than the sensing elements included in the array of sensing elements 210 can be disposed on the substrate 208. In one or more illustrative examples, the additional sensors 220 can include one or more temperature sensors, one or more pressure sensors, one or more humidity sensors, one or more mechanical stress sensors, one or more pH sensors, or combinations thereof. Also, the additional sensors 220 can include one or more photosensors that can measure wavelengths and/or intensity of electromagnetic radiation. For example, the additional sensors 220 can include one or more photodiodes in various situations. In particular implementations, measurements from the additional sensors 220 can be used by the calibration circuitry 212 to determine baseline readings for the sensing elements of the array of sensing elements 210. To illustrate, the calibration circuitry 218 can determine different sets of characteristics for the detection of substances at different environmental conditions, such as various sets of temperature, humidity, mechanical stress, and so forth. Based on a particular set of environmental conditions being experienced by the array of sensing elements 210, the calibration circuitry 218 can cause a specified set of threshold values to be used in the detection of substances by the array of sensing elements 210.

Additionally, sensor protection circuitry 222 can be disposed on the substrate 208. Sensor protection circuitry 222 can operate to monitor and control the amount of usage of sensing elements included in the array of sensing elements 210 in a manner that maximizes the lifetime of the device 202. In one or more examples, the sensor protection circuitry 222 can monitor the amount of usage of sensing elements by monitoring at least one of a number of times that individual sensing elements have been activated or an amount of time that the individual sensing elements have been deactivated. In addition, in various implementations, individual sensing elements of the array of sensing elements 210 can have threshold usage amounts that correspond to a lifetime for the individual sensing elements. For example, individual sensing elements of the array of sensing elements 210 can have limitations on a number of times that the individual sensing elements can be activated and/or limitations on an amount of time that the individual sensing elements can be activated. In these scenarios, the sensor protection circuitry 222 can monitor whether the individual sensing elements have been utilized beyond their lifetime. In situations where a sensing element of the array of sensing elements 210 has met or exceeded a threshold amount of usage, the sensor protection circuitry 222 can cause the sensing element to cease being used to detect one or more substances. In additional implementations where multiple sensing elements included in the array of sensing elements 210 can detect the same one or more substances, the sensor protection circuitry 222 can cause the individual sensing elements used to detect a substance at a given time to be rotated. In this way, the amount of time that each sensing element is used to detect a substance can be extended and can lead to a longer lifetime for the device 202.

Further, in one or more implementations, a protective layer can be disposed over at least a portion of the sensing elements of the array of sensing elements 210. In these implementations, the protective layer can be controlled electrically to limit the exposure of individual sensing elements to the environment in which the device 202 is located. In various examples when sensing elements are to be activated, the sensor protection circuitry 222 can operate independently, or in conjunction with the control circuitry 216, to apply electrical signals to the protective layer of one or more sensing elements of the array of sensing elements 210 in order to modify the protective layer and allow the one or more sensing elements to be exposed to the environment in which the device is located. Additionally, when sensing elements are to be deactivated, the sensor protection circuitry 222 can operate independently, or in conjunction with the control circuitry 216, to cause electrical signals to be absent from the protective layer of one or more sensing elements to enable the protective layer to shield the sensing elements from the environment in which the device 202 is located.

Communication circuitry 224 can be disposed on the substrate 208. The communication circuitry 224 can enable communications to be exchanged between the device 202 and one or more additional devices. In one or more implementations, the communication circuitry 224 can include an interface that enables the device 202 to be physically coupled to an additional device. In one or more illustrative examples, the communication circuitry 224 can include an interface with two power input/output connectors, three digital connectors, and four analog connectors to couple the device 208 with an additional device. In various examples, the communication circuitry 224 can enable the device 202 to be physically coupled to the sensor reader device 204. Additionally, the communication circuitry 224 can include circuitry to enable wireless communications between the device 202 and one or more additional devices. For example, the communication circuitry 224 can include circuitry to enable communications using a wireless local area network, such as a network utilizing an Institute for Electrical and Electronics Engineers (IEEE) 802.11 standard. In one or more additional examples, the communication circuitry 224 can include circuitry to enable communication by the device 202 using near-field communication (NFC) protocols. In further examples, the communication circuitry 224 can include circuitry to enable communications by the device 202 using the Bluetooth communication standard.

Energy storage components 226 can also be disposed on or coupled to the substrate 208. The energy storage components 226 can store energy that can be used by additional components disposed on the substrate 208 to operate the array of sensing elements 210. The energy storage components 226 can include one or more batteries, one or more supercapacitors, or one or more other energy storage devices. In situations where the energy storage components 226 include a battery, the battery can be rechargeable.

The device 202 can be physically or wirelessly coupled to the sensor reader device 204. The sensor reader device 204 can obtain information captured by the device 202 using the array of sensing elements 210. In some examples, the sensor reader device 204 can include a specialized computing device that operates to obtain information from the device 202. In various implementations, the sensor reader device 204 can be a component of a computing device that includes a number of additional components. For example, the sensor reader device 204 can include a mobile computing device, a smart phone, a tablet computing device, a laptop computing device, a desktop computing device, combinations thereof, and so forth. In one or more implementations, the sensor reader device 204 can obtain information from the device 202 indicating one or more substances detected by the array of sensing elements 210. The sensor reader device 204 can also obtain information from the device 202 indicating times that one or more substances were detected by the array of sensing elements 210 and/or environmental conditions under which the one or more substances were detected by the array of sensing elements 210. In one or more implementations, the sensor reader device 204 can obtain information from the device 202 indicating amounts of one or more substances detected by the array of sensing elements 210.

The sensor reader device 204 can store or otherwise have access to the sensor software 206. The sensor software 206 can be executed by the sensor reader device 204, in some implementations, while in additional implementations, one or more additional computing devices can execute the sensor software 206. The sensor software 206 can analyze the information obtained by the sensor reader device 204 from the device 202. In various implementations, the sensor software 206 can be executed to generate user interfaces that indicate information obtained by the sensor reader device 204 from the device 202. For example, the sensor software 206 can generate one or more user interfaces that indicate substances detected by the array of sensing elements 210, amounts of substances detected by the array of sensing elements 210, environmental conditions under which substances were detected by the array of sensing elements 210, timing of detection of substances by the array of sensing elements 210, or combinations thereof. In particular implementations, the sensor software 206 can generate user interfaces that indicate information related to the substances detected by the array of sensing elements 210 gathered over a period of time. In one or more additional implementations, the sensor software 206 can be executed to determine one or more biological conditions that may be associated with substances detected by the array of sensing elements 210.

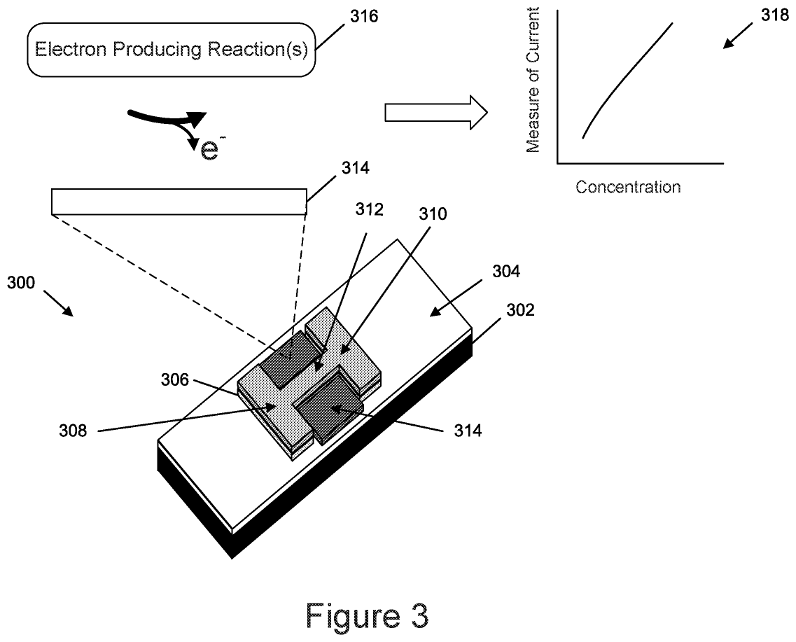

FIG. 3 is a conceptual diagram depicting a sensor 300 that can be included in an integrated sensor array and circuitry in accordance with this disclosure. In the illustrative example of FIG. 3, the sensor 300 is a semiconductor-based sensor. The sensor 300 can be a sensing element included in the array of sensors 104 and/or the array of sensing elements 210. The sensor 300 may include a substrate 302. In one or more examples, the substrate 302 can be a silicon-containing substrate. The sensor 300 can also include a first layer 304 disposed above at least a portion of the substrate 302. In various examples, the first layer 304 can include an oxidation layer. In one or more illustrative examples, the first layer 304 can include a field oxide layer. The sensor 300 may also include a second layer 306. The second layer 306 can include an additional oxide layer in one or more scenarios. In one or more instances, at least one of the first layer 304 or the second layer 306 can be a silicon oxide layer, such as an SiO2-containing layer. In one or more additional implementations, at least one of the first layer 304 or the second layer 306 can be optional. In implementations where the sensor 300 includes the first layer 304 and the second layer 306, the second layer 306 can be disposed over the first layer 304.

Additionally, the sensor 300 can include a source region 308 and a drain region 310. In one or more illustrative examples, the source region 308 and the drain region 310 can include n-type doped regions and the substrate 302 can be a p-type substrate. The sensor 300 can also include a nanowire region 312 that is disposed between the source region 308 and the drain region 310. A silicon-containing nanowire can be included in the nanowire region 312. In one or more implementations, an isolation layer (not shown in FIG. 3) can be disposed over at least one of the source region 308, the drain region 310, or the nanowire region 312. The isolation layer can include an oxide layer. In one or more additional implementations, the isolation layer can include a polymeric layer. In various examples, the sensor 300 can also include a sensing layer 314. The sensing layer 314 can include a polymeric material. In one or more implementations, at least one of the source region 308, the drain region 310, the nanowire region 312, or the sensing layer 314 can include a polysilicon material. In various examples, the source region 308, the drain region 310, and the nanowire region 312 can include a first polysilicon material and the sensing layer 314 can include a second polysilicon material.

In one or more implementations, at least one of the nanowire region 312 or the sensing layer 314 can be functionalized for sensing one or more specified substances included in a sample. For example, an enzyme or other substance that can react with a substance that is to be detected can be disposed on at least one of the nanowire region 312 or the sensing layer 314. In one or more illustrative examples, a material used to functionalize at least one of the nanowire region 312 or the sensing layer 314 can be bonded to atoms of at least one of the nanowire region 312 or the sensing layer 314 via at least one of covalent bonding, ionic bonding, hydrogen bonding, van der Waal's forces, dipole-dipole interactions, or dispersion forces.

In one or more examples, one or more electron producing reactions 316 can take place between one or more substances in a sample. The one or more electron producing reactions 316 can cause an electrical response to take place that can be measured by the sensor 300. For example, the one or more electron producing reactions 316 can cause a change in a measure of current, such as current density, to be produced that can be measured by the sensor 300. In one or more additional examples, the one or more electron producing reactions 316 can cause a change in a measure of voltage to be produced that can be measured by the sensor 300. The electrical response produced by the one or more electron producing reactions 316 can be indicative of a concentration of a substance within a sample. In various examples, as the concentration of a substance increases, the electrical response of the one or more electron producing reactions 316 can increase. In the illustrative example of FIG. 3, as the measure of current detected by the sensor 300 increases, the concentration of a substance being detected also increases. In one or more implementations, the change in the electrical response caused by the one or more electron producing reactions 316 can be detected by a nanowire included in the nanowire region 312.

In one or more illustrative examples, the one or more electron producing reactions 316 can include an oxidation-reduction reaction that produces gluconic acid from glucose in the presence of the enzyme glucose oxidase (GOx). The glucose can be present in a sample that contacts the sensor 300. In one or more examples, GOx can also be present in the sample or the GOx can be bound to a portion of the sensor 300, such as the sensing layer 314. In situations where the GOx is present in the sample, the GOx can be added to an original sample before or during contact of the sample with the sensor 300. In various examples, additional reactants can be added to the sample to cause one or more additional electron producing reactions 316 to take place. In these scenarios, electrons produced by the additional electron producing reactions 316 can be more easily detectable than the electrons produced by the reaction that generates gluconic acid from glucose in the presence of GOx. The presence of electrons produced in response to the one or more electron producing reactions 316 can be detected by the semiconductor-based sensor 300. A number of electrons generated as a result of the one or more electron producing reactions 316 can be used to determine a blood glucose level of an individual.

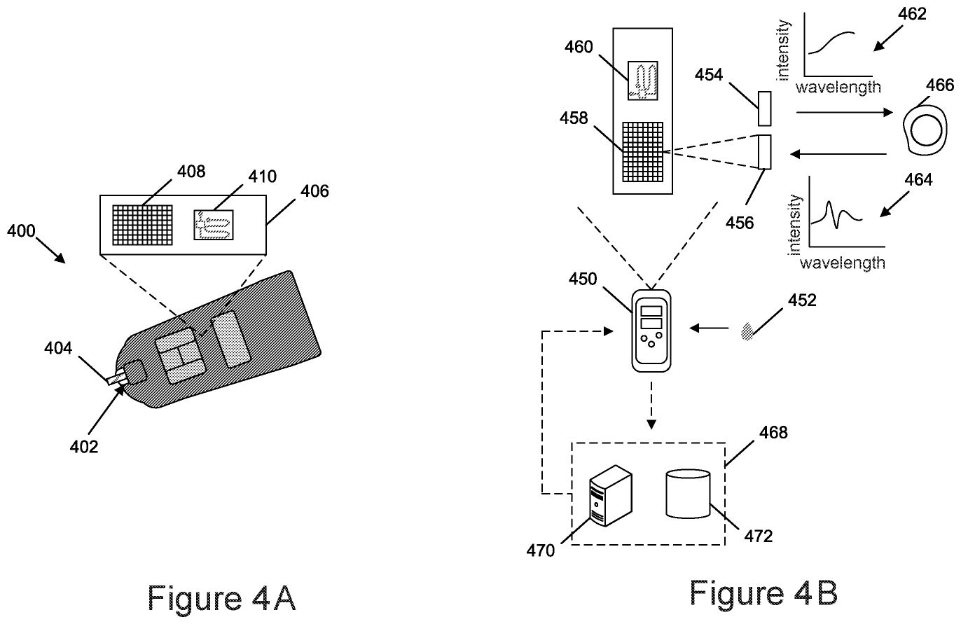

FIG. 4A is a conceptual diagram depicting detection of a substance by contacting the substance with at least one sensor of a sensor array included in a device 400. The illustrative example of FIG. 4A includes a contact sensor device 400. The contact sensor device 400 can include a sample receiving area 402 that can receive a carrier device 404 that includes a sample to be analyzed by the contact sensor device 400. For example, the carrier device 404 can be inserted into the contact sensor device 400 via the sample receiving area 402.

A sample to be analyzed that is included in the carrier device 404 can contact an integrated sensor device 406 that includes a sensor array 408 and circuitry 410. In one or more examples, the integrated sensor device 406 can include at least one of the device 100 of FIG. 1 or the device 202 of FIG. 2. After the carrier device 404 is inserted into the sample receiving area 402 and the sample contacts at least one sensor of the sensor array 408, the at least one sensor can generate signals that can indicate at least one substance included in the sample. In various examples, at least a portion of the sensors in the sensor array 408 can be configured to produce an electrical response when contacted with a sample that includes the substance.

The circuitry 410 can control the operation of the sensor array 408 with respect to the detection of one or more substances included in a sample. For example, the circuitry 410 can activate and deactivate one or more sensors of the sensor array 408. In addition, the circuitry 410 can operate to store data corresponding to signals generated by the sensor array 408 in at least one of memory of the contact sensor device 400 or memory that is located remotely with respect to the contact sensor device 400. In various examples, the circuitry 410 can communicate data to a system that analyzes the signals generated by the sensor array 408 and provide results of the analysis for display via the contact sensor device 400. The results of the analysis can indicate at least one of the presence or absence of one or more substance in a sample on the carrier device 404. In one or more additional examples, the contact sensor device 400 can analyze the signals generated by the sensor array 408 to determine results of the analysis. In one or more examples, the circuitry 410 can analyze, at least in part, the signals produced by the sensor array 408 to identify at least one of the presence or absence of one or more substances included in a sample on the carrier device 404. The circuitry 410 can include at least a portion of at least one of the first circuitry 106 or the second circuitry 108. The circuitry 410 can also include at least a portion of the circuitry disposed on the substrate 208, such as at least a portion of at least one of the first switch circuitry 212, the second switch circuitry 214, the control circuitry 216, the calibration circuitry 218, circuitry related to the one or more additional sensors 220, the sensor protection circuitry 222, the communication circuitry 224, or circuity related to the energy storage components 226.

FIG. 4B is a conceptual diagram depicting detection of a substance by at least one sensor of a sensor array included in a device 450 without contacting the substance with the at least one sensor. The device 450 can analyze a sample 452 to determine whether one or more substances are included in the sample 452. The device 450 can detect the presence or absence of one or more substances using a non-contact-based process that analyzes changes to electromagnetic radiation that is applied to the sample 452.

In one or more examples, the device 450 can include one or more emitters 454 that emit one or more ranges of wavelengths of electromagnetic radiation and one or more detectors 456 that detect electromagnetic radiation emitted by the one or more emitters 454. At least one of the one or more emitters 454 or the one or more detectors 456 can be included in a sensor array 458 of the device 450. In various examples, the one or more emitters 454 can be included in the circuitry 460. The one or more emitters 454 can emit electromagnetic radiation having a profile 462 that has an intensity value for a given wavelength value over a range of wavelengths. In situations where the electromagnetic radiation emitted by the one or more emitters 454 interacts with a substance 466 included in the sample 452, an additional profile 464 can be produced that is different from the initial profile 462. The wavelengths and corresponding intensities associated with the additional profile 464 can be detected by the one or more detectors 456. That is, the initial profile 462 of electromagnetic radiation emitted by the one or more emitters 454 can be modified by the substance 464.

The additional profile 464 can then be analyzed to identify the substance 466. In one or more illustrative examples, the additional profile 464 can be analyzed with respect to one or more template profiles that have been previously determined for one or more substances. The one or more template profiles can indicate changes to the initial profile 462 in response to interaction with the one or more substances. The analysis of the additional profile 464 can be performed, at least in part, by the circuitry 460. In one or more additional examples, the analysis of the additional profile 464 can be performed, at least in part, by a system 468 that is located remotely from the device 450. The system 468 can include one or more processing devices 470 and one or more data storage devices 472. After analyzing the additional profile 464, the device 450 can display an indication of the presence or absence of one or more substances. In one or more illustrative examples, the device 450 can display an indicator of the presence of the substance 466 in the sample 452. By analyzing profiles of the emission and detection of electromagnetic radiation, the device 450 can detect the presence or absence of one or more substances without the sample 452 contacting the sensor array 458.

The circuitry 460 can control the operation of the sensor array 458 with respect to the detection of one or more substances included in the sample 452. For example, the circuitry 460 can activate and deactivate one or more sensors of the sensor array 458. In addition, the circuitry 460 can operate to store data corresponding to signals generated by the sensor array 458 in at least one of memory of the device 450 or memory that is located remotely with respect to the device 450. The circuitry 460 can include at least a portion of at least one of the first circuitry 106 or the second circuitry 108 of FIG. 1. The circuitry 460 can also include at least a portion of the circuitry disposed on the substrate 208, such as at least a portion of at least one of the first switch circuitry 212, the second switch circuitry 214, the control circuitry 216, the calibration circuitry 218, circuitry related to the one or more additional sensors 220, the sensor protection circuitry 222, the communication circuitry 224, or circuity related to the energy storage components 226 of FIG. 2.

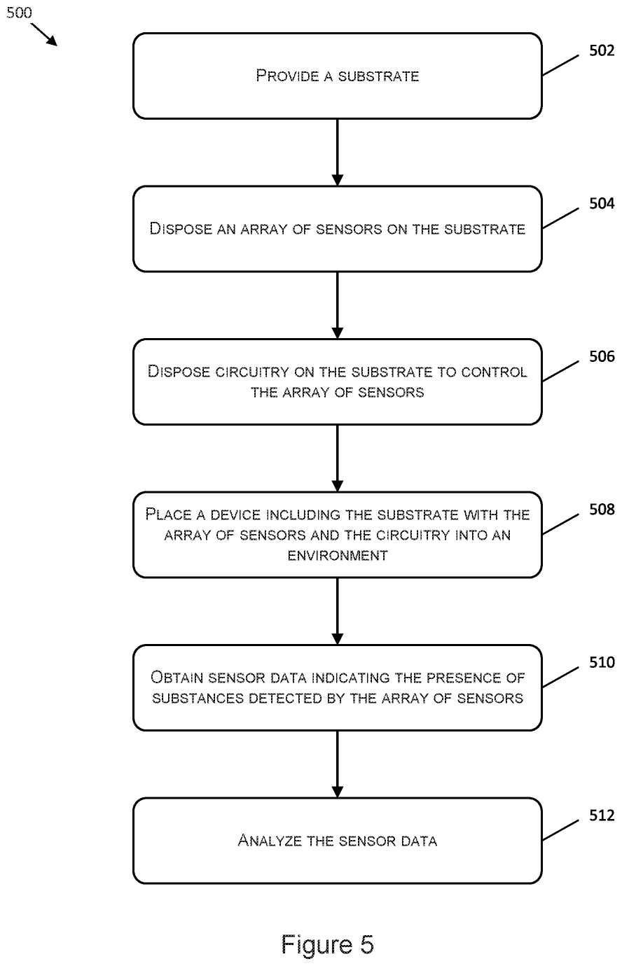

FIG. 5 is a flow diagram depicting an example of operations of a process 500 to produce and use an integrated sensor array and circuitry in accordance with this disclosure. At 502, the process 500 can include providing a substrate. The substrate can be relatively rigid and formed from materials such as silicon or glass, in particular implementations. In additional implementations, the substrate can be relatively flexible and formed from one or more polymeric materials, such as a polyamide, a polyethylene terephthalate, or from a paper material.

At 504, the process 500 can include disposing an array of sensors on the substrate. The array of sensors can include semiconductor devices that are formed on the substrate using semiconductor related processes, such as lithography operations, doping operations, etching operations and rinsing operations. The array of sensors can include, in various implementations, carbon nanotubes formed on the substrate or wires formed on the substrate, such as gold or platinum wires having diameters no greater than 250 nm.

At 506, the process 500 can include disposing circuitry on the substrate to control the array of sensors. The circuitry can include logic, switches, connectors, and other components. The circuitry can be disposed on the substrate using semiconductor processing operations. In implementations, the circuitry can include components to control the operation of the sensors included in the array of sensors. For example, the circuitry can operate to activate and deactivate the sensors included in the array of sensors. In addition, the circuitry can include components to calibrate the array of sensors and circuitry to enable communication of information produced by the array of sensors and the circuitry to one or more external devices. Further, the circuitry can include memory devices, energy storage devices, and additional sensors, such as a temperature sensor, a pH sensor, a moisture sensor, a pressure sensor, a mechanical stress sensor, a light sensor, or one or more combinations thereof.

At 508, the process 500 can include placing a device including the substrate with the array of sensors and the circuitry into an environment. In various implementations, after the array of sensors and the circuitry are formed on the substrate, the combination of the substrate, circuitry, and array of sensors can comprise a device. In some implementations, the device can be placed into a housing. In illustrative examples, the device can be placed into an environment and individual sensors included in the sensor array can detect the presence of substances in the environment. In particular examples, the device can be placed into a liquid environment or a gaseous environment. Additionally, the substances detected by the array of sensors included in the device can include substances found in liquids, substances found in solids, substances found in gases, or combinations thereof.

At 510, the process 500 can include obtaining sensor data indicating the presence of substances detected by the array of sensors. The data can indicate the composition of the substances and/or a quantity of the substances. In some cases, the sensor data can correspond to signals produced by individual sensors included in the array of sensors in response to detecting the substances. In one or more implementations, the sensor data can be obtained via a sensor reader device.

At 512, the process 500 can include analyzing the sensor data. The analysis of the sensor data can determine substances located in the environment based on the detection of the substances by the array of sensors. The analysis of the sensor data can also determine a quantity of the substances located in the environment. In addition, analyzing the sensor data can determine timing information related to the presence of the substance in the environment. In various implementations, the analysis of the sensor data can be used to generate one or more user interfaces that can indicate one or more metrics derived from the sensor data.

Each of the non-limiting aspects or examples described herein may stand on its own or may be combined in various permutations or combinations with one or more of the other examples.

The above detailed description includes references to the accompanying drawings, which form a part of the detailed description. The drawings show, by way of illustration, specific embodiments in which the invention may be practiced. These implementations are also referred to herein as “examples.” Such examples may include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein may be machine or computer-implemented at least in part. Some examples may include a computer-readable medium or machine-readable medium encoded with instructions operable to configure an electronic device to perform methods as described in the above examples. An implementation of such methods may include code, such as microcode, assembly language code, a higher-level language code, or the like. Such code may include computer readable instructions for performing various methods. The code may form portions of computer program products. Further, in an example, the code may be tangibly stored on one or more volatile, non-transitory, or non-volatile tangible computer-readable media, such as during execution or at other times. Examples of these tangible computer-readable media may include, but are not limited to, hard disks, removable magnetic disks, removable optical disks (e.g., compact discs and digital video discs), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments may be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments may be combined with each other in various combinations or permutations. The scope of the invention should be determined with reference to the appended claims, along with the full scope of equivalents to which such claims are entitled.

### Example Aspects

Aspect 1. An apparatus comprising: a substrate; an array of sensors disposed on the substrate, individual sensors of the array of sensors having a dimension that is no greater than 750 nanometers (nm); and circuitry electronically coupled to the array of sensors and disposed on the substrate, the circuitry to activate or deactivate at least one sensor of the array of sensors.

Aspect 2. The apparatus of aspect 1, wherein individual sensors of the array of sensors are arranged in a grid including a first number of columns and a second number of rows, first sensors included in an individual column of the first number of columns are electrically coupled via a first connector, and second sensors included in an individua row of the second number of rows are electrically coupled via a second connector, the second connector being disposed substantially perpendicular with respect to the first connector; and wherein the apparatus comprises: first circuitry disposed on the substrate, the first circuitry being coupled to the first connector and the first circuitry including a first plurality of switches; and second circuitry disposed on the substrate, the second circuitry being coupled to the second connector and the second circuitry including a second plurality of switches.

Aspect 3. The apparatus of aspect 2, further comprising control circuitry disposed on the substrate and coupled to the first circuitry and the second circuitry, the control circuitry to cause at least one switch of the first plurality of switches and at least one switch of the second plurality of switches to operate to activate a sensor of the array of sensors.

Aspect 4. The apparatus of aspect 3, wherein the control circuity is configured to cause the at least one switch of the first plurality of switches and the at least one switch of the second plurality of switches to operate to deactivate the sensor of the array of sensors.

Aspect 5. The apparatus of any of aspects 1-4, comprising one or more additional sensors disposed on the substrate, the one or more additional sensors including at least one of a temperature sensor, a pressure sensor, a pH sensor, a mechanical stress sensor, a moisture sensor, or an electromagnetic radiation sensor.

Aspect 6. The apparatus of any of aspects 1-5, wherein the substrate includes a silicon-containing material or a glass-containing material.

Aspect 7. The apparatus of any of aspects 1-5, wherein the substrate includes a polymeric material including at least one of a polyamide, a polyethylene terephthalate, or a paper material.

Aspect 8. The apparatus of any of aspects 1-8, wherein the individual sensors of the array of sensors include at least one of a semiconductor-based sensor, a carbon nanotube-based sensor, or a wire-based sensor.

Aspect 9. The apparatus of aspect 8, wherein the semiconductor-based sensor includes an n-type ZnO-containing field effect transistor, a p-type Ge-containing field effect transistor, an n-type Ge-containing field effect transistor, a p-type Si-containing field effect transistor, or an n-type Si-containing field effect transistor.

Aspect 10. The apparatus of aspect 8 or 9, wherein the semiconductor-based sensor includes a fin field effect transistor (FET), a bioFET, or an ion-sensitive FET.

Aspect 11. The apparatus of aspect 8, wherein the wire-based sensor includes a wire having a diameter no greater than 250 nm and formed from at least one Au, an Au-containing alloy, Pt, or a Pt-containing alloy.

Aspect 12. The apparatus of any of aspects 1-11, wherein the array of sensors includes a first number of sensors to detect a first substance and a second number of sensors to detect a second substance.

Aspect 13. The apparatus of any of aspects 1-11, wherein the array of sensors includes a plurality of sensors to detect a substance, the plurality of sensors includes a first sensor that is enabled to detect the substance, and the apparatus comprises additional circuitry to: detect an amount of use of a first sensor of the plurality of sensors, the amount of use including at least one of a number of activations of a first sensor of the plurality of sensors or an amount of time that the first sensor has been in an activated state; determine that the amount of use of the first sensor is at least a threshold amount of use; disable the first sensor with respect to detection of the substance; and enable a second sensor of the plurality of sensors to detect the substance.

Aspect 14. The apparatus of any of aspects 1-12, comprising further circuitry to: determine one or more first baseline characteristics corresponding to detection of the substance by a first sensor of the array of sensors; and determine one or more second baseline characteristics corresponding to detection of the substance by a second sensor of the array of sensors, wherein the one or more second baseline characteristics are different from the one or more first baseline characteristics.

Aspect 15. The apparatus of any of aspects 1-14, comprising communication circuitry to transmit first signals to one or more first devices according to at least one wireless communication standard and receive second signals from the one or more second devices according to the at least one wireless communication standard.

Aspect 16. The apparatus of any of aspects 1-15, comprising an interface to physically couple the apparatus to an additional device.

Aspect 17. The apparatus of any of aspects 1-16, comprising an energy storage device including at least one of a battery or a supercapacitor.

Aspect 18. The apparatus of any of aspects 1-17, wherein the dimension includes at least one of a width, a length, or a diameter.

Aspect 19. A system comprising: a device including: a substrate; an array of sensors disposed on the substrate, wherein sensors included in the array of sensors are arranged in a grid including a first number of columns and a second number of rows and individual sensors of the array of sensors have at least one of a width, a length, or a diameter that is no greater than 750 nanometers (nm); a first number of switches individually coupled to individual columns of the first number of columns; a second number of switches individually coupled to individual rows of the second number of rows; and circuitry to cause activation of a sensor of the array of sensors, the sensor being located at an intersection of a column of sensors included in the first number of columns and a row of sensors included in the second number of rows, and wherein the circuitry activates the sensor by applying a first electrical signal to the sensor via a first switch coupled to the column of sensors and by applying a second electrical signal to the sensor via a second switch coupled to the row of sensors.

Aspect 20. The system of aspect 19, comprising a sensor reader device to obtain data from the device, the data corresponding to one or more substances detected by the array of sensors.

Aspect 21. The system of aspect 20, comprising a computing device including at least one hardware processor and memory, the memory storing computer-readable instructions that, when executed by the at least one hardware processor, perform operations comprising: performing an analysis of the data corresponding to the one or more substances detected by the array of sensors.

Aspect 22. A method comprising: providing a substrate, the substrate being formed from a silicon-containing material, a glass containing material, or a polymeric material; disposing an array of sensors on the substrate, the array of sensors including sensors arranged in a grid including a first number of columns and a second number of rows and individual sensors of the array of sensors have at least one of a width, a length, or a diameter that is no greater than 750 nanometers (nm); and disposing circuitry on the substrate, the circuitry to cause at least one of activation or deactivation of a sensor included in the array of sensors.

Aspect 23. The method of aspect 22, comprising: placing the device in an environment; detecting, by at least one sensor of the array of sensors, a substance in the environment; and obtaining data from the device indicating that the substance is included in the environment.

1. An apparatus comprising:
a substrate;
an array of sensors disposed on the substrate, individual sensors of the array of sensors having a dimension that is no greater than 750 nanometers (nm); and
circuitry electronically coupled to the array of sensors and disposed on the substrate, the circuitry to activate or deactivate at least one sensor of the array of sensors.

2. The apparatus of claim 1, wherein individual sensors of the array of sensors are arranged in a grid including a first number of columns and a second number of rows, first sensors included in an individual column of the first number of columns are electrically coupled via a first connector, and second sensors included in an individua row of the second number of rows are electrically coupled via a second connector, the second connector being disposed substantially perpendicular with respect to the first connector; and wherein the apparatus comprises:
first circuitry disposed on the substrate, the first circuitry being coupled to the first connector and the first circuitry including a first plurality of switches; and
second circuitry disposed on the substrate, the second circuitry being coupled to the second connector and the second circuitry including a second plurality of switches,

3. The apparatus of claim 2, further comprising control circuitry disposed on the substrate and coupled to the first circuitry and the second circuitry, the control circuitry to cause at least one switch of the first plurality of switches and at least one switch of the second plurality of switches to operate to activate a sensor of the array of sensors.

4. The apparatus of claim 3, wherein the control circuity is configured to cause the at least one switch of the first plurality of switches and the at least one switch of the second plurality of switches to operate to deactivate the sensor of the array of sensors.

5. The apparatus of claim 1, comprising one or more additional sensors disposed on the substrate, the one or more additional sensors including at least one of a temperature sensor, a pressure sensor, a pH sensor, a mechanical stress sensor, a moisture sensor, or an electromagnetic radiation sensor.

6. The apparatus of claim 1, wherein the substrate includes a silicon-containing material or a glass-containing material.

7. The apparatus of claim 1, wherein the substrate includes a polymeric material including at least one of a polyamide, a polyethylene terephthalate, or a paper material.

8. The apparatus of claim 1, wherein the individual sensors of the array of sensors include at least one of a semiconductor-based sensor, a carbon nanotube-based sensor, or a wire-based sensor.

9. The apparatus of claim 8, wherein the semiconductor-based sensor includes an n-type ZnO-containing field effect transistor, a p-type Ge-containing field effect transistor, an n-type Ge-containing field effect transistor, a p-type Si-containing field effect transistor, or an n-type Si-containing field effect transistor.

10. The apparatus of claim 8, wherein the semiconductor-based sensor includes a fin field effect transistor (FET), a bioFET, or an ion-sensitive FET.

11. The apparatus of claim 8, wherein the wire-based sensor includes a wire having a diameter no greater than 250 nm and formed from at least one Au, an Au-containing alloy, Pt, or a Pt-containing alloy.

12. The apparatus of claim 1, wherein the array of sensors includes a first number of sensors to detect a first substance and a second number of sensors to detect a second substance.

13. The apparatus of claim 1, wherein the array of sensors includes a plurality of sensors to detect a substance, the plurality of sensors includes a first sensor that is enabled to detect the substance, and the apparatus comprises additional circuitry to:
detect an amount of use of a first sensor of the plurality of sensors, the amount of use including at least one of a number of activations of a first sensor of the plurality of sensors or an amount of time that the first sensor has been in an activated state:
determine that the amount of use of the first sensor is at least a threshold amount of use;
disable the first sensor with respect to detection of the substance; and
enable a second sensor of the plurality of sensors to detect the substance.

14. The apparatus of claim 1, comprising further circuitry to:
determine one or more first baseline characteristics corresponding to detection of the substance by a first sensor of the array of sensors; and
determine one or more second baseline characteristics corresponding to detection of the substance by a second sensor of the array of sensors, wherein the one or more second baseline characteristics are different from the one or more first baseline characteristics.

15. The apparatus of claim 1, comprising communication circuitry to transmit first signals to one or more first devices according to at least one wireless communication standard and receive second signals from the one or more second devices according to the at least one wireless communication standard.

16. The apparatus of claim 1, comprising:
an interface to physically couple the apparatus to an additional device: and
an energy storage device including at least one of a battery or a supercapacitor.

17. (canceled)

18. (canceled)

19. A system comprising:
a device including:
a substrate:
an array of sensors disposed on the substrate, wherein sensors included in the array of sensors are arranged in a grid including a first number of columns and a second number of rows and individual sensors of the array of sensors have at least one of a width, a length, or a diameter that is no greater than 750 nanometers (nm);
a first number of switches individually coupled to individual columns of the first number of columns;
a second number of switches individually coupled to individual rows of the second number of rows; and
circuity to cause activation of a sensor of the array of sensors, the sensor being located at an intersection of a column of sensors included in the first number of columns and a row of sensors included in the second number of rows, and wherein the circuitry activates the sensor by applying a first electrical signal to the sensor via a first switch coupled to the column of sensors and by applying a second electrical signal to the sensor via a second switch coupled to the row of sensors.

20. The system of claim 19, comprising:
a sensor reader device to obtain data from the device, the data corresponding to one or more substances detected by the array of sensors: and
a computing device including at least one hardware processor and memory, the memory storing computer-readable instructions that, when executed by the at least one hardware processor, perform operations comprising performing an analysis of the data corresponding to the one or more substances detected by the array of sensors.

1. (canceled)

22. A method comprising:
providing a substrate, the substrate being formed from a silicon-containing material, a glass containing material, or a polymeric material;
disposing an array of sensors on the substrate, the array of sensors including sensors arranged in a grid including a first number of columns and a second number of rows and individual sensors of the array of sensors have at least one of a width, a length, or a diameter that is no greater than 750 nanometers (nm); and
disposing circuitry on the substrate, the circuitry to cause at least one of activation or deactivation of a sensor included in the array of sensors.

23. The method of claim 22, comprising
placing the device in an environment;
detecting, by at least one sensor of the array of sensors, a substance in the environment; and
obtaining data from the device indicating that the substance is included in the environment.
